(19) United States
(12) Patent Application Publication
(10) Pub. No.: US 2013/0266909 A1
(43) Pub. Date: Oct. 10, 2013

(54) IMPLANT FOR USE IN A PHOTODYNAMIC TREATMENT

(71) Applicants: Daniel Delfosse, Jegenstorf (CH); Thomas Oberbach, Reichenbach (DE); Henning Glasser, Niederzimmern (DE)

(72) Inventors: Daniel Delfosse, Jegenstorf (CH); Thomas Oberbach, Reichenbach (DE); Henning Glasser, Niederzimmern (DE)

(21) Appl. No.: 13/822,203

(22) PCT Filed: Sep. 9, 2011
(86) PCT No.: PCT/EP2011/065619

§ 371 Date: Jun. 27, 2013

(30) Foreign Application Priority Data
Sep. 10, 2010 (DE) 10 2010 045 061.8
Nov. 10, 2010 (DE) 10 2010 050 898.5
May 13, 2011 (DE) 10 2011 075 808.9

(51) Int. Cl. A61N 5/06; B05D 3/06

(52) U.S. Cl. CPC A61N 5/062; B05D 3/06
USPC 433/173; 604/20; 604/501; 427/2.26

(57) ABSTRACT

An implant (10) for implantation in a human or animal bone (11) has a bone area (12), which is in contact with the bone (11), and a light area, which is not covered by the bone (11). It likewise has a photo-activatable substance (14), which is activated when illuminated with light (2) and thereafter destroys microbes and bacteria. The implant (10) is made of a material which is transparent at, at least, one activation wavelength of the photo-activatable substance (14), wherein the photo-activatable substance (14) is applied at least to the surface of the bone area (12) of the implant.

The invention relates to an implant for implantation in a human or animal bone, comprising a bone region that is in contact with the bone and a light region that is not covered by the bone, the implant being suitable for use with a photodynamic treatment. In addition, the invention relates to a method for producing an interface between the bone and the implant.

At present, metallic implants made of titanium or titanium alloys are frequently employed in the field of dental prosthetics. For example, DE 197 13 012 A1 describes a dental implant, which is composed of an implantable implant body, made of titanium, for example, and a structural part, which is screwed into a thread of the implant body. The structural part itself is likewise made of titanium or a titanium alloy and is surrounded by a ceramic body. Dental implants that are produced solely from ceramics are also used increasingly. The advantage of ceramics is the better aesthetic effect, especially in the anterior tooth region, and better adhesion of the soft tissue, such as the gingiva, to the implant.

One problem in odontology, especially after implanting an implant, is periimplantitis. This is caused by bacteria and bacterial plaque, which are found in the natural oral flora/fauna and become embedded in the interface between the bone and implant during implantation. In the long run, this results in inflammation of this interface and prevents the lasting integration of the implant.

Bacterial infections are also possible with hip, shoulder, knee or intervebral disk implantations, for example as a result of so-called nosocomial germs, and are occurring with increasing frequency due to ever more frequent, complicated and difficult surgeries as well as complicated instrument-based, invasive measures. When such bacterial infections occur in the dental field or also in the other described fields, an antibacterial treatment is required, which until now has often only been possible by way of a revision surgery, which is to say removal of the implant. The ever more prevalent use of antibiotics additionally results in increasing resistance of various bacterial strains to antibiotics or even in antibiotic intolerance.

It is the object of the invention to create an implant that allows treatment by way of photodynamic therapy for a potential bacterial infection during or after implantation.

The object is achieved by the inventive implant according to claim 1. A method for producing a corresponding interface with the implant is described in claim 12. The dependent claims describe advantageous refinements of the implant according to the invention.

The implant according to the invention for implantation in a human or animal bone comprises a bone region that is in contact with the bone and a light region that is not covered by the bone. It also comprises a photo-activatable substance, which is activated when irradiated with light and subsequently destroys microbes and bacteria. The implant is produced from a material that is transparent at least at an activation wavelength of the photo-activatable substance, wherein the photo-activatable substance is at least applied to the surface of the bone region of the implant.

Due to the transparency of the implant to at least the activation wavelength, it is possible to illuminate the implant, and in particular the photo-activatable substance, applied to the bone region of the implant, even after insertion into the animal or human bone, and thereby activate the antibacterial effect thereof. This is easily possible in the case of a dental implant because the implant is not completely covered by a dental prosthesis until the bone has fully healed, or at least this prosthesis can be easily removed. The activation of the photo-activatable substance applied prior to implantation is thus possible at different time intervals. If the photo-activatable substance is depleted, it can be reapplied, for example by way of an injection.

In the case of implants that are not directly accessible, such as acetabular cup, shoulder joint or intervertebral disk implants, light, and more particularly light generated by a laser, can be introduced by way of arthroscopic surgery using an optical waveguide via the light region of the implant. A treatment with antibiotics and the revision surgery of the implant are thus not necessary. This poses a considerably lesser burden for the patient and saves the costs of a revision operation and a new implant.

The implant is advantageously produced from a ceramic material because ceramic material, being a bioinert prosthesis material, has extremely favorable properties with respect to compatibility in the human body, has low abrasion and is extremely resistant to breakage. In addition, implants made of a ceramic material have been successfully applied in artificial joint replacement for a long time.

It is particularly advantageous for the ceramic material to be composed of a zirconium oxide ceramic material, an aluminum oxide ceramic material or a dispersion ceramic material as a mixture of zirconium oxide and aluminum oxide. The ceramic material is advantageously stabilized by yttrium oxide, cerium oxide, calcium oxide, magnesium oxide or other oxides. This increases the strength of the material and reduces cracks when the ceramic material cools after sintering.

The ceramic material is advantageously produced from a nanopowder, the particles of which have a size smaller than 500 mn. In particular, particle sizes smaller than 200 nm are used, so that an extremely fine structure having particles measuring smaller than 500 nm is created during sintering. The particle size is thus below the wavelength that is irradiated so as to activate the photo-activatable substance and thus allows this light to pass. Light, which is generated by a laser, for example, can thus pass through the implant and activate the photo-activatable substance applied to the surface in the bone region.

The photo-activatable substance is advantageously applied by spraying the substance onto the implant or by immersing the implant in the photo-activatable substance.

The method according to the invention for producing an interface between a bone and an implant with a photo-activatable substance employs an implant comprising a bone region that is in contact with the bone and a light region that is not covered by the bone. The implant is made of a material that is transparent at least to the activation wavelength of the photo-activatable substance and is coated on the surface of the bone region with a photo-activatable substance. Light having the activation wavelength is introduced into the implant via the light region and activates the photo-activatable substance at the interface between the bone region of the implant and the bone. After the implant has been inserted into the bone, an antibacterial treatment can thus be carried out, without requiring the use of antibiotics, and in particular without having to remove the implant.

Exemplary embodiments of the implant of the method according to the invention are shown by way of example in the drawings and will be described hereafter in greater detail. In the drawings:

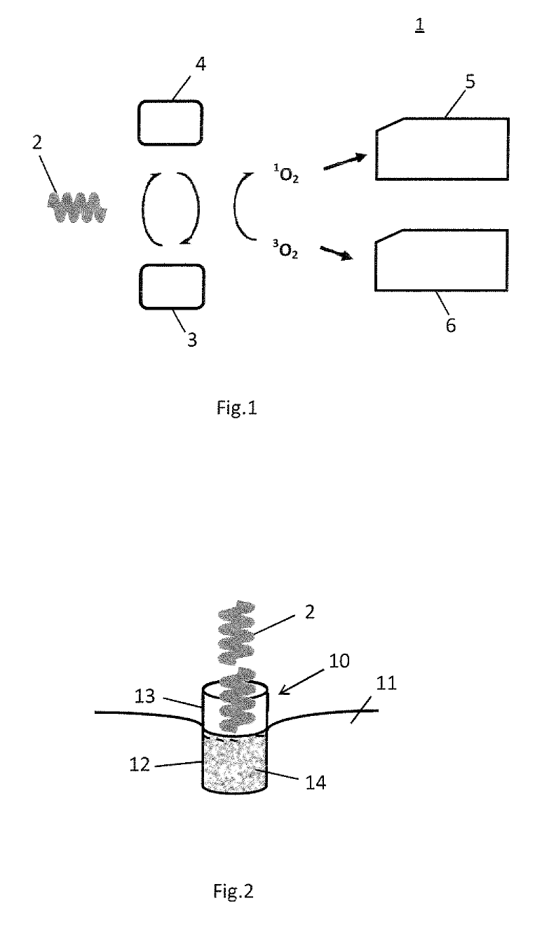

FIG. 1 is a schematic illustration of a mechanism of action of a photodynamic treatment;

FIG. 2 is a schematic illustration of an exemplary embodiment according to the invention of a dental implant;

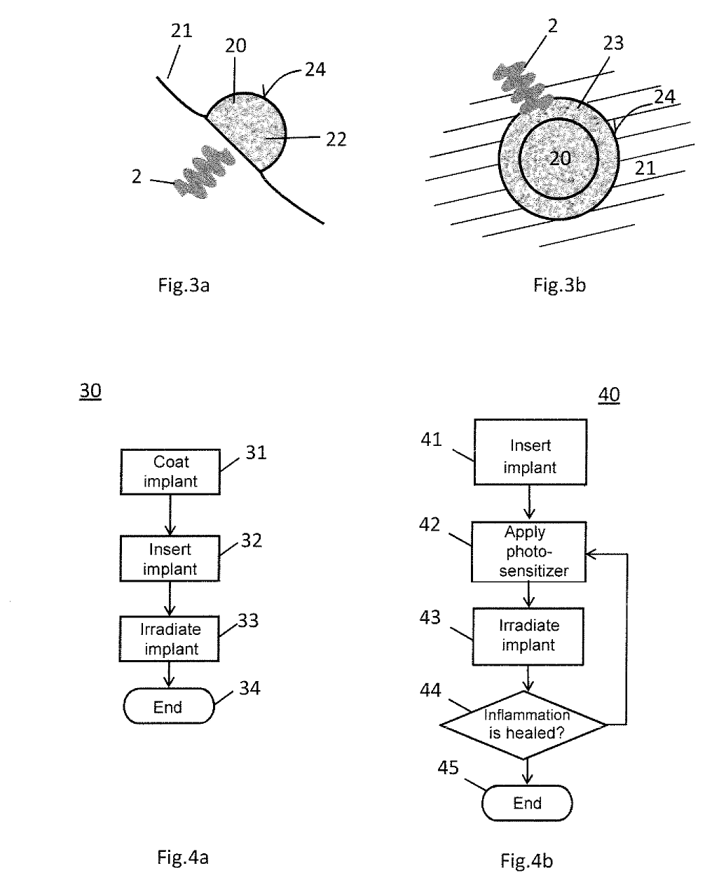

FIG. 3a is a schematic illustration of an exemplary embodiment according to the invention of an acetabular cup implant;

FIG. 3b is a top view onto the light region of the acetabular cup implant according to the invention shown in FIG. 3a;

FIG. 4a is a block diagram of a first exemplary embodiment of a treatment method according to the invention; and

FIG. 4b is a block diagram of a second exemplary embodiment of a treatment method according to the invention during healing.

Like parts are denoted by like reference numerals in all the figures.

FIG. 1 is a schematic illustration of the mechanism of action of a photodynamic treatment. For this purpose, a photo-activatable substance, also referred to as a photosensitizer, is brought in contact with human tissue, in which oxygen molecules are usually present in a variety of ways.

Upon irradiation of the photosensitizer, this substance in particular absorbs light 2 having a particular wavelength, this being the so-called activation wavelength, and transitions into a first excited singlet state. Upon further irradiation, this singlet photo-sensitizer 3 transitions into a triplet state by way of intercombination, resulting in an excited photosensitizer 4. Because the energy of the excited sensitizer molecule 4 is greater than the energy that is required for oxygen to transition into an excited singlet state, this exchange of energy can take place. The resulting singlet oxygen damages cell constituents in the vicinity because of the chemical reactivity thereof and causes intracellular oxidation 5. This, in turn, leads to necrosis, which is to say a destruction of the cells 6.

When erythrosine B or safranin O is used as the photosensitizer, a targeted destruction of bacteria can be achieved because these substances, when used for Gram staining, deposit on Gram-positive and/or Gram-negative bacteria.

This photochemical reaction is utilized for the antibacterial treatment at the contact surface between the implant and the bone. FIG. 2 shows this based on the example of a dental implant 10, which is implanted into a jaw bone 11. The implant 10 has a light region 13 protruding over the bone 11 and a bone region 12 in contact with the bone 11.

The dental implant 10, itself, is made of a transparent ceramic material, preferably a transparent zirconium oxide ceramic material, which is reinforced with aluminum oxide and stabilized with yttrium oxide. To produce such a transparent ceramic material, the green body is produced from a nanopowder having a particle size smaller than 500 nm, and more particularly smaller than 200 nm. The nanopowder itself is produced by way of a synthetic method and, after being treated with water, binding agent and additives so as to obtain a slip, is shaped into a green body by way of pressing or using a wet shaping method, such as a slip casting or pressure slip casting method. The structure resulting after sintering has particle sizes that are smaller than the activation wavelength and thus allow the activation wavelength to pass.

The photo-activatable substance 14 is applied to the bone region 12 of the implant 10. Such a coating process can be carried out by spraying the photo-activatable substance onto the bone region 12 or by immersing the bone region 12 into the photo-activatable substance 14. This substance is preferably present as a photosensitizer-containing gel or a photosensitizer-containing solution. After the implant 10 has been anchored in the jaw bone 11 and primary fixation has been conducted, the irradiation with laser light having a defined wavelength is carried out. Said photosensitizers erythrosine B and safranin O have an activation wavelength of approximately 530 nm, and more particularly 532 nm.

The activation wavelength of most photosensitizers is in the visible spectral range, in particular in the red spectral range from approximately 630 nm to 750 nm.

The laser light source is placed directly on the light region 13 for irradiation. The light beam 2 passes through the transparent ceramic implant 10 and exits the implant 10 from within the bone region 12. The light impinges on the photo-activatable substance 14 located on the surface of the bone region 12. The photo-activatable substance, which is connected to the cell envelope of the microbes and/or is received in the microbe, is excited by the light 2. The described photochemical process is triggered. The irreversible change of bacterial components results in the selective destruction of the microbes and, consequently, in a considerable reduction of the microbial count causing the infection. The infection is reduced or stopped.

Activation by way of laser light can also be carried out in the course of the healing process after implantation if an acute infection develops. In this case, the bone region of the implant coated with the photo-activatable substance is likewise irradiated with light 2 having the activation wavelength. In the case of a dental implant 10, this can be easily applied from the outside, by removing a temporary crown that may have been attached. In the case of transparent ceramic implants that do not allow direct access from the outside, such as a hip, shoulder or intervertebral disk implant, light 2 is introduced by way of arthroscopic surgery, for example, using an optical waveguide, via the light region of the implant.

FIGS. 3a and 3b show such a transparent ceramic acetabular cup implant 20. FIG. 3a shows a sectional view of a transparent ceramic hip implant 20 that is inserted into a hip bone 21. The photo-activatable substance 24 is applied in a bone region 22 of the acetabular cup implant 20, which corresponds approximately to the entire outer surface of the acetabular cup implant 20 facing the bone.

FIG. 3b is a top view onto the same implant 20. The bone region 22 coated with photo-activatable substance 24 is not visible here or is only visible in a phantom view of the implant. Only the uncoated light region of the acetabular cup 20 is visible. This is embedded in the hip bone 21 shown with dotted lines. The light 2 is now irradiated, for example through an optical waveguide, which is placed on the light region, in particular on the cup edge 23. Light 2 passes through the transparent acetabular cup implant 20 and/or is reflected at the interface with the femoral head or at the interface with the hip bone and impinges on the photo-activatable substance 24, where it triggers the described photochemical effect. This allows bacteria or microbes in the interface region between the implant 20 and bone 21 to be destroyed.

Diagram 30 in FIG. 4a shows the individual steps of a photodynamic treatment applied to a transparent ceramic implant 10, 20. In the first step 31, a transparent implant according to the invention, which is provided with a photo-activatable substance 14, 24 in a bone region 12, 22, is coated. In the next step 32, this transparent implant 10, 20 is inserted into a bone 11, 21. In step 33, light 2 having the activation wavelength is irradiated at the light region 13, 23 of the implant 10, 20, either already during implantation or also not until a later time. The light 2 passes through the transparent implant 10, 20 and impinges on the photo-activatable substance 14, 24, which is applied to the surface in the bone region 12, 22 of the implant.

The bacteria are killed in the tissue that adjoins the bone region 12, 22 and has come in contact with the photo-activatable substance. Thereafter, in step 34, the treatment is completed. If necessary, the activation by way of light, see step 33, can be repeated, in particular, if the implant 10, 20 was coated with a larger quantity of photo-activatable substance 14, 24, which was not exclusively activated during a first irradiation and therefore depleted.

FIG. 4b shows a method for the treatment with photo-activatable substances, in particular for implants that are surrounded at least partially by softer tissue. As a prerequisite for this step, a transparent implant, for example a dental implant 10, is inserted in step 41, and the surrounding tissue demonstrates an inflammatory reaction. So as to combat bacterial inflammation, photo-activatable substance 14, for example in gel form or as an aqueous solution, is applied in step 42 to the surface of the bone region 12 of the implant 10. In the case of a dental implant 10, this is done through the gingiva by way of injection, for example. In step 43, a potential cover of the implant 10 is removed and light 2 having the activation wavelength is irradiated via the light region 13 of the implant. If thereafter, in step 44, it is ascertained that the inflammation has healed, the treatment can be ended, see step 45. If the inflammation is still present, the photodynamic treatment can be repeated by repeating method steps 42 and 43.

All of the features described and/or shown can advantageously be combined with each other within the scope of the invention. The invention is not limited to the shown exemplary embodiments.

1-16. (canceled)

17. An implant for implantation in a human or animal bone, comprising
a bone region (12, 22) that is in contact with the bone (11, 21);
a light region (13, 23) that is not covered by bone (11, 21); and
a photo-activatable substance (14, 24), which is activated when illuminated with light (2) having an activation wavelength and then destroys microbes and bacteria;
wherein the implant (10, 20) comprises a material that is transparent at least at the activation wavelength of the photo-activatable substance.

18. The implant according to claim 17,
characterized in that
the photo-activatable substance is at least applied to the bone region (12, 22) of the implant (10, 20).

19. The implant according to claim 17,
characterized in that
the implant (10, 20) is at least partially composed of a ceramic material.

20. The implant according to claim 19,
characterized in that
the ceramic material is a zirconium oxide ceramic material or an aluminum oxide ceramic material or a ceramic material made of a mixture of zirconium oxide and aluminum oxide.

21. The implant according to claim 20,
characterized in that
the ceramic material is stabilized with Y₇O₃, CeO₂, CaO, MgO or other oxides.

22. The implant according to claim 19,
characterized in that
the ceramic material is produced from nanopowder having a particle size smaller than 500 nm, in particular smaller than 200 nm.

23. The implant according to claim 17,
characterized in that
the applied photo-activatable substance (14, 24) is erythrosine B and/or safranin O.

24. The implant according to claims 17,
characterized in that
the photo-activatable substance (14, 24) is applied by spraying the substance onto the implant (10, 20) or by immersing the implant (10, 20) in the photo-activatable substance (14, 24).

25. The implant according to claim 17,
characterized in that
the photo-activatable substance (14, 24) is present in a gel or in a solution.

26. The implant according to claims 17,
characterized in that
the implant (10) is a dental implant for insertion into a jaw bone (11).

27. An implant according to claim 7,
characterized in that
the implant is a hip joint, shoulder joint or vertebral column implant.

28. A method for producing an interface between a bone (11, 21) and an implant (10, 20) with a photo-activatable substance (14, 24),
wherein the implant (10, 20) comprises a bone region (12, 22) that is in contact with the bone (11, 21) and a light region (13, 23) that is not covered by bone (11, 21),
wherein the implant (10, 20) is produced from a material that is at least transparent to an activation wavelength of the photo-activatable substance (14, 24).

29. The method according to claim 28,
characterized in that
a photo-activatable substance (14, 24) is applied at least to the surface of the bone region (12, of the implant (10, 20).

30. The method according to claim 28,
characterized in that
light having the activation wavelength is irradiated into the implant (10, 20) via the light region (13, 23) and the photo-activatable substance (14, 24) at an interface between the bone region (12, 22) of the implant (10, 20) and the bone is thereby activated.

31. The method according to claim 30,
characterized in that
the photo-activatable substance (14, 24) is produced dissolved in a gel or ire a solution.

32. The method according to claim 30,
characterized in that
light (2) is applied by illumination with a laser.
